(19) United States
(12) Patent Application Publication
(10) Pub. No.: US 2022/0211285 A1
(43) Pub. Date: Jul. 7, 2022

(54) DIAGNOSIS

(71) Applicants: Rajeshwar MOOKERJEE, London (GB); Rajiv JALAN, London (GB)

(72) Inventors: Rajeshwar MOOKERJEE, London (GB); Rajiv JALAN, London (GB)

(21) Appl. No.: 17/601,378

(22) PCT Filed: Apr. 6, 2020
(86) PCT No.: PCT/GB2020/050899

§ 371 Date: Oct. 4, 2021

(30) Foreign Application Priority Data
Apr. 5, 2019 (GB) 1904861.0

(51) Int. Cl. A61B 5/024; A61B 5/00; A61B 5/0205

(52) U.S. Cl. CPC A61B 5/02405; A61B 5/0205; A61B 5/7275; A61B 5/0006

(57) ABSTRACT

The present invention relates to a method of predicting acute decompensation (AD) and/or the development of acute-on-chronic liver failure (ACLF) in a patient having the condition, or at risk of having the condition, using heart-rate variability (HRV) as a marker.

### FIELD OF THE INVENTION

The present invention derives from the finding that continuous, wireless remote monitoring of heart rate variablilty HRV is feasible in patients with acute decompensation of cirrhosis, acute-on-chronic liver failure (ACLF), or those at risk of developing ACLF, and measuring standard deviation of all normal beat-to-beat variation of R-R intervals (SDNN) reflects changes in HRV in this population.

The inventors found that SDNN is reduced significantly with acute decompensation of cirrhosis (which progresses in a matter of days to weeks) and even further reduced in patients with ACLF.

A reduction in SDNN, reflecting loss of HRV, negatively (and significantly) correlates with (liver) disease severity scores in acute decompensation such as Model for End-Stage Liver Disease (MELD) and Child-Pugh (CP) scores, CLIF-C AD score and importantly, with systemic inflammation.

In addition, measurement of HRV, for example, by measuring SDNN, shows utility in predicting early (e.g. from about 28-day up to 90-day) mortality after acute cirrhosis decompensation or in patients at risk of developing ACLF. A SDNN cut-off of ≥12 ms has a negative predictive value of 93% in determining outcome. Moreover, patients who represent to the hospital within 3-4 months following prior successful hospital discharge, invariably have SDNN<20 ms suggesting that thresholds of SDNN may guide need for early intervention in patients at risk of cirrhosis decompensation.

### BACKGROUND TO THE INVENTION

Some patients with cirrhosis (who may have been stable for a significant period of time) can suddenly deteriorate and manifest as the discrete entity of acute decompensation in days to weeks, and to date, there are no clear factors that predict this decompensation. Acute decompensation (herein referred to as AD) may result from many precipitants including, bacterial infection, large-volume ascites, GI haemorrhage, or hepatic encephalopathy, alone or in combination, and in many cases, no obvious precipitant may be attributable. Such AD patients are at risk of further decompensation to organ failure and have an increased morbidity and mortality compared to stable outpatient cirrhosis. Furthermore, in about 25% of patients, the AD presentation may be with rapidly progressive hepatic and/or extra-hepatic organ failure, a syndrome referred to as acute-on-chronic liver failure (ACLF). About 20% of these patients progress to multi-organ failure and death.

There are currently no specific treatments for ACLF beyond supportive management and in very select cases, consideration for urgent liver transplantation is their only salvage treatment.

End stage cirrhosis accounts for 40,000 deaths in the US— nearly as many as diabetes mellitus and 170,000 patients across EU nations. In Europe, about 640,000 are admitted to the hospital. As 20-30% of these are likely to have some degree of organ failure and thereby are at risk of further deterioration with ACLF, risk of mortality is high and for those that recover, re-admission to the hospital is common with about 33% readmitted within one month of discharge, at a cost of $20,000 per admission. It follows that an early diagnosis of acute decompensation and progression to ACLF, with a sensitive marker to predict early deterioration, would help to improve timely intervention, with clear health economic benefits.

Under normal physiological conditions, cardiac responses to physical activity and stress (increased cardiac output) are regulated largely by the autonomic nervous system. The interplay between sympathetic and parasympathetic autonomic nervous system activity is reflected in heart rate variability (HRV), the variation in normal heart beat-beat intervals, with greater parasympathetic modulation promoting increased HRV in a linear manner. Loss of normal HRV is a feature of systemic inflammation and increasing age.

Patients with cirrhosis are known to have numerous cardiac abnormalities including prolongation of the QTc interval and altered baroreceptor responses. Moreover, a reduction in HRV has been described in patients with cirrhosis and it has been suggested that a further loss occurs with increasing severity of cirrhosis, and in the presence of hepatic encephalopathy. However, such observations have been largely based in stable cirrhosis out-patients with limited 5-minute electrocardiography (ECG) assessments. It is well established that AD and ACLF are distinct clinical entities within the cirrhosis spectrum. Therefore, while studies into HRV have been performed in the past within the stable cirrhosis patients, quantitative and significant changes in HRV with acute decompensation of cirrhosis and development of acute-on-chronic liver failure (ACLF) and the prognostic significance of any such changes, has to date been uncharacterised. There remains a need for a diagnostic and prognostic method which allows for monitoring of patients with AD and/or ACLF, and for those that recover from such an episode, at risk of further AD and/or ACLF development.

### SUMMARY OF THE INVENTION

The present invention is based on the finding that progressive loss of heart rate variability (HRV) is a feature of acute decompensation of cirrhosis and progression in acute-on-chronic liver failure (ACLF) and, surprisingly, predicts early mortality of patients with acute decompensation and ACLF. Early mortality herein refers to e.g. a 90-day mortality, or preferably a 28-day to 90-day mortality.

The present inventors found that continuous, wireless remote monitoring of HRV is feasible in patients with acute decompensation and ACLF. Furthermore, loss of HRV, denoted by lower baseline standard deviation of all normal beat-to-beat variation of R-R intervals (SDNN), correlated with severity of acute decompensation (AD). In addition, SDNN was significantly lower in patients developing ACLF, or at risk of developing ACLF, compared to those with only AD and correlated inversely with Model for End-Stage Liver Disease (MELD), and Child-Pugh scores, and inflammatory indices such as C-reactive protein (CRP) and white cell count. SDNN predicted disease progression on repeat measures and was the only independent predictor of 90-day mortality; a cut-off of >12 ms having a 93% negative predictive value. More than 67% patients readmitted to the hospital following a successful discharge in the prior 3-4 months, had an SDNN <19.65 ms.

HRV remote monitoring identifies patients at high risk of progression to ACLF and death, and shows that HRV monitoring can be used to guide early intervention in such patients.

This invention proposes methods for a novel acute decompensation of cirrhosis care management system for patients at home to improve (short-term) outcomes and reduce the current costs and morbidity from acute decompensation of cirrhosis and ACLF. The invention envisages the use of individualized patient data through measurement of HRV acquired through a wireless physiological sensor platform, and uses given thresholds of SDNN combined with defined disease modelled algorithms of care, to deliver a personalized treatment plan for patients at risk of acute decompensation of cirrhosis and ACLF in the community.

As such the invention provides:
A method of predicting the severity of acute decompensation of cirrhosis or acute-on-chronic liver failure (ACLF) in a patient having the condition, or at risk of having the condition, using heart-rate variability (HRV) as a marker.
A database comprising information relating to HRV and its association with the likelihood of 90-day mortality in patients having, or at risk of having, AD or ACLF.
A method of determining the likelihood of 90-day mortality in a patient having, or at risk of having, AD or ACLF, comprising:
(a) inputting to a computer system data concerning the HRV of the patient;
(b) comparing the data to a computer database, which database comprises information relating to HRV and its association with the likelihood of 90-day mortality in patients having, or at risk of having, AD or ACLF; and
(c) determining on the basis of the comparison the likelihood of 90-day mortality in the patient or a future acute decompensation event.
A computer program comprising program code means that, when executed on a computer system, instruct the computer system to perform all the steps of the method of the invention.
A computer storage medium comprising the computer program according to the invention and the database according to the invention.
A computer system arranged to perform the method according to the invention, comprising:
(a) means for receiving data concerning the HRV of patients with, or at risk of having, AD or ACLF;
(b) a database comprising information relating to HRV and its association with the likelihood of 90-day mortality in patients with, or at risk of having, AD or ACLF;
(c) a module for comparing the data with the database; and
(d) means for determining on the basis of said comparison the likelihood of 90-day mortality in the patients.
A method of monitoring the severity of AD or ACLF in a patient, comprising the use of a device capable of remotely monitoring the HRV of the patient.
A method of monitoring a patient with acute decompensation of cirrhosis or acute-on-chronic liver failure (ACLF) for prognosis of survival, which comprises:
(a) obtaining an initial measurement for heart rate variability (HRV);
(b) obtaining one or more subsequent measurements of HRV of the patient and determining whether said subsequent measurements fit any of predetermined criteria showing progression and/or severity of cirrhosis or acute-on-chronic liver failure (ACLF);
further optionally comprising a step (c) outputting an alarm if said measurements reaches a predetermined threshold for intervention.

### BRIEF DESCRIPTION OF THE DRAWINGS

FIG. 1: Stable outpatients with cirrhosis have reduced HRV compared to healthy controls. The development of acute decompensation (AD) results in significantly lower range of SDNN, then in stable cirrhosis.

FIG. 2A: As patients develop organ failure and evolve to ACLF, there is an even greater derangement of HRV denoted by further reduction of SDNN vs. those patients who remain as AD without developing ACLF.

FIG. 2B: HRV levels correlate inversely with measures of liver disease progression such as the Child Pugh and MELD scores.

FIG. 3: Representative Poincare plots showing progressive loss of HRV from a control healthy individual (A), to a patient with outpatient stable cirrhosis (B). Further loss of HRV is demonstrated with acute decompensation of cirrhosis (C) and this is most marked in a patient with ACLF pre-mortem (D).

FIG. 4: Baseline SDNN demonstrates good predictive utility for overall mortality within 3 months in patients with, or at risk of developing, AD, with an area under receiver operator curve of 0.75. There is a negative predictive value for death by 90 days of 93% for a SDNN of ≥12 ms.

FIG. 5: A Kaplan-Meier analysis in which a cut-off for SDNN <12 ms identifies those patients with markedly greater 3 month mortality of 40% vs. only 8% in those with SDNN≥12 ms, emphasizing the utility of an SDNN thresholds to highlight AD patients at risk of poor outcome

FIG. 6: Representative plots of acute decompensated cirrhosis patients with repeated measures of SDNN over 5 days, during their episode of acute decompensation. Failure to increase SDNN from low baseline values or further SDNN loss with time, is associated with high risk of death.

FIG. 7: Baseline SDNN shows strong inverse correlation with markers of inflammation including C-reactive protein (CRP) and white cell count (WCC). There are similar trends in correlations with inflammatory cytokines e.g. Interleukin-6 and interleukin-8.

### DETAILED DESCRIPTION OF THE INVENTION

It is to be understood that different applications of the disclosed methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural references unless the content clearly dictates otherwise. Thus, for example, reference to “a patient” includes “patients”, and the like.

All publications, patents and patent applications cited herein, whether supra or infra, are hereby incorporated by reference in their entirety.

Acute decompensation of cirrhosis (AD) defined by the acute development of one or more major complications of liver disease (i.e. ascites, encephalopathy, gastrointestinal haemorrhage, bacterial infection), is the main cause of hospitalization in patients with cirrhosis. AD develops in many cirrhotic patients, in some cases in the absence of a defined precipitating event. In some cases, it is associated with organ failure(s) (ie, worsening of liver function and/or kidney failure and/or failure of other organs). Patients with cirrhosis hospitalized for an acute decompensation (AD) and organ failure are at risk for imminent death are considered to have acute-on-chronic liver failure (ACLF).

Acute-on-chronic liver failure (ACLF) is a distinct clinical entity encompassing an acute deterioration of liver function in patients with cirrhosis, often decompensated cirrhosis, which is usually associated with a precipitating event and results in the failure of one or more organs and high short term mortality. Unregulated inflammation is thought to be a major contributing factor. A characteristic feature of ACLF is its rapid progression, the requirement for multiple organ supports and a high incidence of short and medium term mortality of 50-90%. ACLF mortality is associated with loss of organ function and high leukocyte counts.

ACLF is diagnosed by use of the Chronic Liver Failure (CLiF) Consortium criteria, NACSELD criteria or APASL criteria. Previously validated scores to assess disease severity include Child-Pugh (CP) classification, Model for End Stage Liver Disease (MELD) and the CLiF Consortium Acute Decompensation (CLIF-C AD) score.

Severity in ACLF can be defined as progression from ACLF grade 1, to ACLF grade 2 and finally to ACLF grade 3. ACLF grade 1: This group includes 3 subgroups: (1) patients with single kidney failure, (2) patients with single failure of the liver, coagulation, circulation, or respiration who had a serum creatinine level ranging from 1.5 to 1.9 mg/dL and/or mild to moderate hepatic encephalopathy, and (3) patients with single cerebral failure who had a serum creatinine level ranging from 1.5 and 1.9 mg/dL. The 90-day mortality is approximately 40%. ACLF grade 2. This group includes patients with 2 organ failures. The 90-day mortality approximately 50%. ACLF grade 3. This group includes patients with 3 organ failures or more. The 90-day mortality rate is approximately 80%.

Increased severity in ACLF can be defined as an increased likelihood of 90-day mortality in comparison to a patient with AD but not ACLF.

### HRV

Under normal physiological conditions, cardiac responses to physical activity and stress (increased cardiac output) are regulated largely by the autonomic nervous system. The interplay between sympathetic and parasympathetic autonomic nervous system activity is reflected in heart rate variability (HRV), the variation in normal heart beat-beat intervals, with greater parasympathetic modulation promoting increased HRV in a linear manner.

Numerous factors affect HRV measurement including respiration, intrinsic cardiac abnormalities, circadian rhythm, age and genetic factors. Moreover, given significant inter- and intra-individual differences in HRV, reliable measurement of HRV has historically been deemed complex and requisite of specific conditions and necessitating special equipment. The present inventors have found that continuous, ambulatory, remote measurement of HRV in patients with ACLF, or at risk of developing ACLF, in whom autonomic dysfunction is common, is possible.

Traditional ECG measurement of HRV encompasses short-term 5 minutes ECG segments being interpreted as reflecting HRV at that specific time, under stable physiological conditions. The remote monitoring system used in an embodiment of the present invention, by contrast, not only facilitates continuous monitoring irrespective of the individuals' daily activity or physical ill health but also helps negate the short coming of limited ECG time capture, where artifact and premature beats caused by these factors over the 5 minutes of analyzed R-R interval, make further interpretation difficult.

This is particularly the case in patients at risk of ACLF, in whom high respiratory rates, inflammation and impaired baroreceptor responses interfere considerably with standard ECG R-R interpretation. Using the remote monitoring device, the present inventors were able to accommodate artifact and interpret HRV in 96% of the patients studied, irrespective of disease severity, aetiology or reason for decompensation.

HRV data can be described in both time and frequency domain variables. Standard deviation of all normal beat-to-beat variation of R-R intervals (SDNN) is the most commonly described HRV time domain measurement in part, through its simple evaluation without the need for complex analytical systems, unlike frequency domain variables. In addition, many frequency domain measures are influenced by high respiratory rate (common in cirrhosis patients) and gender, and necessitate significant short-term data filtering. As a consequence, in a preferred embodiment of the invention, HRV is monitored by measuring SDNN.

### Prediction of the Likelihood of Early Mortality

The methods of the present invention allow the prediction of the severity of ACLF in a patient having, or at risk of having, ACLF. The methods of the present invention allow the prediction of the likelihood of early mortality in a patient having, or at risk of having, ACLF. Measurement of HRV by the methods of the present invention, for example, by measuring SDNN, shows utility in predicting early mortality in patients with AD or ACLF or those at risk of developing AD or ACLF. Moreover, assessing HRV upon discharge, defines patients at risk of re-admission to the hospital with further decompensation. In a preferred embodiment of the invention, an SDNN value, or cut off, of 20 ms or less, 18 ms or less, 16 ms or less, 14 ms or less, 13 ms or less, 12 ms or less, 11 ms or less, or 10 ms or less, is indicative of early mortality in a patient with, or at risk of having, AD or ACLF. In a preferred embodiment of the invention the SDNN cut-off of is 12 or less.

Early mortality herein is defined as a 90-day mortality or preferably a 28-day to a 90-day mortality.

Measurement of HRV can be by any method known on the art. Measurement of SDNN can be by any means known in the art.

### A Method of Monitoring

The present invention also provides a method of monitoring a patient with acute decompensation of cirrhosis or acute-on-chronic liver failure (ACLF) for prognosis of survival, which comprises:

(a) obtaining an initial measurement for heart rate variability (HRV);
(b) obtaining one or more subsequent measurements of HRV of the patient and determining whether said subsequent measurements fit any of predetermined criteria showing progression and/or severity of cirrhosis or acute-on-chronic liver failure (ACLF);
further optionally comprising a step (c) outputting an alarm if said measurements reaches a predetermined threshold for intervention.

Also provided is a computer program which implements the methods of monitoring as provided herein.

As shown in the examples herein, the present invention is based on a novel finding of direct correlation between the severity of acute decompensation of cirrhosis or acute-on-chronic liver failure (ACLF) in a patient and a measurement of HRV, preferably using SDNN. It has been demonstrated that SDNN is not only an indicator of mortality within a short (˜3-month period, for example 28 day to 90 day period), but that monitoring of SDNN can inform an overall prognosis, including the favourability of the patient's response to therapy and/or whether further intervention is required. In some examples, interventions as used herein may refer to one or more of clinical readmission, further monitoring, or further therapeutic intervention.

Thus in such methods of monitoring, it is preferred that the monitoring is conducted continuously and/or in real time. In one example, the method comprises the use of a device capable of remotely monitoring HRV in the patient. Such devices may be wireless. Such devices may be configured to implement the methods of monitoring as provided herein.

In one example, SDNN is used as the measurement of HRV.

In one example, the predetermined criteria may indicate progression and an increase in severity and be one or more of:
a) a decrease in SDNN compared to the starting value, preferably wherein the decrease in SDNN is equal to or more than 2 ms, 3 ms, 4 ms or 5 ms from the starting value;
b) a percentage decrease in SDNN compared to the starting value, preferably wherein the decrease in SDNN is equal to or more than 5%, 6%, 7%, 8%, 9%, 10%, 11%, 12%, 13%, 14% or 15% of the starting value;
c) a measurement of SDNN within a threshold range of SDNN, preferably wherein the threshold range has an upper SDNN limit of: 25 ms, 24 ms, 23 ms, 22 ms or 21 ms, and a lower SDNN limit of: 13 ms, 14 ms, 15 ms, 16 ms, 17 ms, 18 ms, or 19 ms;
d) a measurement of SDNN equal to or below a threshold limit for SDNN, preferably wherein the threshold limit is 19 ms, 18 ms, 17 ms, 16 ms or 15 ms; more preferably wherein the threshold limit is 19 ms.

In one example, the predetermined criteria may indicate a reduction in severity and be one or more of:
a) an increase in SDNN compared to the starting value, preferably wherein the increase in SDNN is equal to or more than 2 ms, 3 ms, 4 ms or 5 ms from the starting value;
b) a percentage increase in SDNN compared to the starting value, preferably wherein the increase in SDNN is equal to or more than 5%, 6%, 7%, 8%, 9%, 10%, 11%, 12%, 13%, 14% or 15% of the starting value;
c) a measurement of SDNN equal to or above a threshold limit for SDNN, preferably wherein the threshold limit is 20 ms, 21 ms, 22 ms, 23 ms, 24 ms.

In one example, the predetermined threshold is one or more of the predetermined criteria.

### Remote Monitoring Devices

In the assessment of HRV in states such as AD and ACLF, in which acute alterations in physiology and inflammation modify day-to-day changes in HRV, continuous monitoring to track such dynamic changes is preferred.

In a preferred embodiment of the present invention, monitoring of HRV is conducted remotely. In a preferred embodiment of the present invention, the remote monitoring of HRV is by a wireless monitoring system. Such remote monitoring systems are known in the art.

The Isansys Lifetouch® system is an example of a remote monitoring system useful for the present invention. The system allows continuous, wireless collection of heart rate and cardiac performance data, sampling at a rate of 1000 Hz, using a simple non-invasive skin surface sensor, which attaches to the precordium and communicates with an accompanying wireless gateway. The heart beat-beat intervals recorded by the device can then be calculated through automatic QRS identification and extracted from the gateway following the recording period.

### Bioinformatics

Information relating to HRV, for example, SDNN, may be stored in an electronic format, for example in a computer database. Accordingly, the invention provides a database comprising information relating to HRV and its association with the likelihood of 90-day mortality in patients with, or at risk of having, ACLF.

The database may also comprise information relating to any of the other markers of AD or ACLF, progression of AD or ACLF, severity of ACLF, as described herein. The database may include further information on HRV or other markers of AD or ACLF, for example the degree of association of the marker with the likelihood of 90-mortality in patients with, or at risk of having, AD or ACLF, or likelihood of readmission with new decompensation of cirrhosis.

A database as described herein may be used to determine the severity of AD or ACLF in a patient with AD. Such a determination may be carried out by electronic means, for example by using a computer system (such as a PC).

A database as described herein may be used to determine the likelihood of 90-day mortality in a patient with, or at risk of having, AD or ACLF. Such a determination may be carried out by electronic means, for example by using a computer system (such as a PC).

Typically, the determination of the likelihood of 90-day mortality in a patient with, or at risk of having, AD or ACLF will be carried out by inputting to a computer system HRV data from the patient to a computer system; comparing the data to a database as defined herein; and on the basis of this comparison, determining the likelihood of 90-day mortality in the patient. This information can then be used to guide the management and intervention in the patient with, or at risk of having, AD or ACLF.

The invention also provides a computer program comprising program code means for performing all the steps of a method of the invention when said program is run on a computer. Also provided is a computer program product comprising program code means stored on a computer readable medium for performing a method of the invention when said program is run on a computer. A computer program product comprising program code means on a carrier wave that, when executed on a computer system, instruct the computer system to perform a method of the invention is additionally provided.

### Treatment Regimens

The methods of the invention can be used to guide the management of treatment in the patient with, or at risk of having, AD or ACLF. The methods of the invention may include a step of providing treatment of the patient with, or at risk of having, AD or ACLF, based on the results of the prognostic methods of the invention described herein. Providing treatment to the patient with, or at risk of having, AD or ACLF may involve regular monitoring of the patient at hospital or in the community. Providing treatment to the patient with, or at risk of having, AD or ACLF may involve administering a therapeutic agent or combination of agents to the patient, to treat their AD or ACLF, such as targeted anti-microbial therapy, stopping or modulating dosage of diuretic therapy, administering albumin infusions or future use of anti-inflammatory targeted therapy (e.g. TLR4 antagonist and or interventions targeting non-apoptotic cell death). The results of the prognostic methods of the invention may determine whether the patient with, or at risk of having, AD or ACLF is provided with therapeutic or palliative care.

### Examples

### Methods

Patients with an established diagnosis of cirrhosis based on clinical, ultrasound and/or laboratory/histological criteria of cirrhosis were included at the Liver and Transplantation Unit, Royal Free hospital, London. The study was approved by the local governing Research Ethics Committee (London Harrow; REC Ref: 08/H0714/8) and all patients (or a family member if without capacity) provided informed consent in accordance with the 1975 Helsinki Declaration. Patients were recruited prospectively between March 2013 and July 2015 at the Royal Free Hospital, London, as part of an on-going biomarker study of cirrhosis decompensation. Patients were excluded if: hospitalised for reasons other than a liver related decompensation of cirrhosis; hospitalized with established ACLF; they had underlying malignancy including hepatocellular carcinoma; they had significant cardiac chronotropic (including atrial fibrillation) or inotropic abnormalities or/and cardiac pacemakers; they had undergone major surgery or were pregnant. Patients taking negative chronotropic medications such as beta-blockers were also excluded.

### Follow Up of the Patients

All patients had clinical, demographic and laboratory parameters recorded at baseline with further serial measures at regular intervals during their period of follow up. All patients underwent a baseline ECG before initiating continuous HRV assessment as described below.

The Chronic Liver Failure (CLiF) Consortium criteria were used to define the development of ACLF during admission in AD patients. Previously validated scores including Child-Pugh (CP), Model for End Stage Liver Disease (MELD) and the CLiF Consortium Acute Decompensation (CLIF-C AD) score were applied to assess disease severity. During follow-up, evolution of disease scores, development of organ failure, ACLF and death were recorded.

All patients underwent routine clinical care with management according to local hospital guidelines and directed by the treating clinicians. This included intensive care unit (ICU) and specific organ support, such as renal filtration, when indicated. Patients were observed until hospital discharge with further follow-up data collected to 90 days.

### Heart Rate Variability Evaluation

A total of 42 patients were assessed for HRV by wireless remote monitoring (LifeTouch® gateway). Five minute, artefact-free, continuous R-R interval sessions, during the waking hours of 8 am and 7 pm, were selected for analysis using Kubios HRV Version 2.0, to derive SDNN values for a given subject for that corresponding day. HRV data from the wireless remote device was also compared with standard ECG/Holter R-R interval recording derived SDNN, and showed good concordance with these measurements.

The patients included 35 patients with acute decompensation (AD) of cirrhosis defined by acute rise in bilirubin to values >5× upper limit of normal, or/and new onset of ascites, or presence of renal dysfunction or hepatic encephalopathy. Infection or alcoholic hepatitis were common precipitants of decompensation. AD patients [median CLIF-AD score was 54 (range 38-84)], were compared with 7 stable cirrhosis outpatients (O/P) with no ascites or organ dysfunction and with bilirubin <3× upper limit of normal.

Patients with AD were followed during admission and the development of acute-on-chronic liver failure (as defined by the criteria of the Cannonic study—Moreau et al, Gastroenterology 2013) was noted in 9 patients. HRV was re-assessed in these patients at the onset of ACLF.

### Plasma IL-6 and IL-8 Measurement

Interleukin (IL)-6 and IL-8 were measured using standard ELISA kits as per manufacturer's instruction (BD Biosciences).

### Example 1—Relationship of SDNN with Acute Decompensation and ACLF

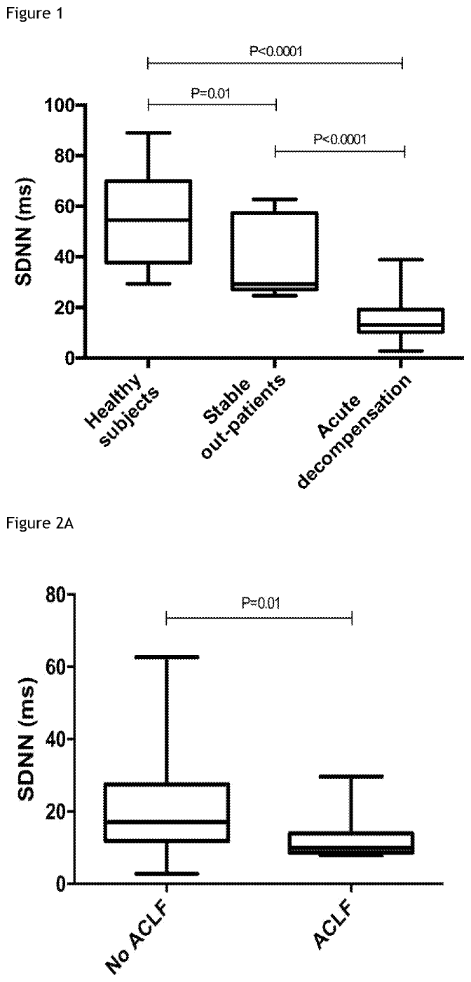

Stable outpatient cirrhosis patients have reduced HRV compared to healthy controls and is related to Child classification (Child C having lower values then Child B and A). However, a striking observation was that the development of acute decompensation (AD) resulted in markedly lower SDNN range, than stable cirrhosis [AD: 13 (10-19) vs. O/P: 29 (27-57); median and IQR; P=0.0004] (FIG. 1).

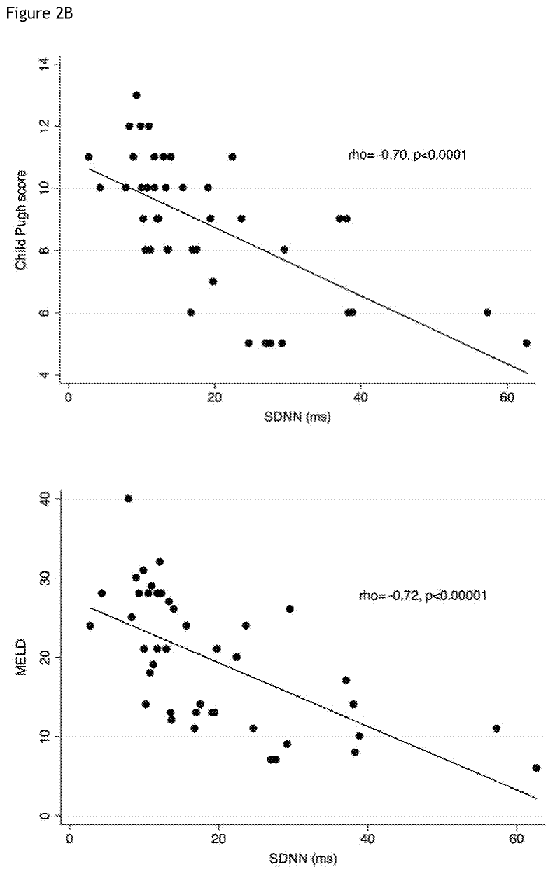

Furthermore, as patients develop organ failure and evolve to ACLF, there was an even greater derangement of HRV denoted by SDNN [AD progression to ACLF: 10 (9-14) vs. those patients who remain as AD: 14 (11-20), P=0.04] (FIG. 2A).

The progression of liver disease as denoted by increasing Child-Pugh and MELD scores is inversely correlated with loss of SDNN as shown in FIG. 2B.

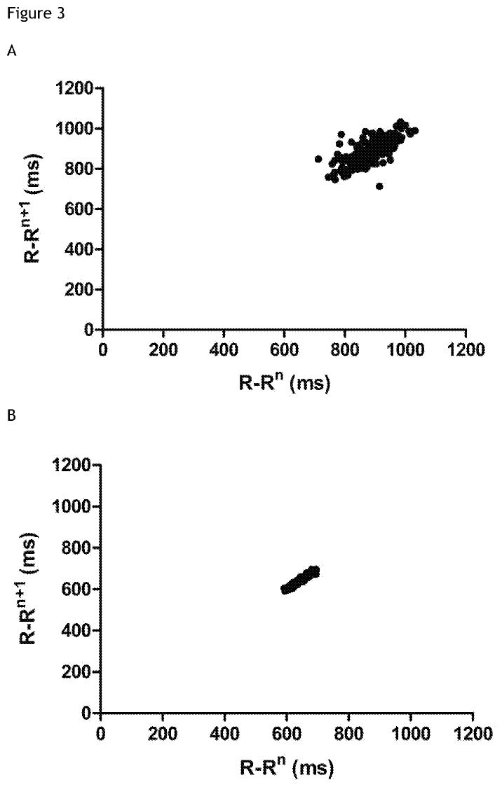

Representative Poincare plots were created from a control healthy individual (FIG. 3A), to a patient with outpatient stable cirrhosis (FIG. 3B) showing this progressive loss of HRV. Further loss of HRV was demonstrated with acute cirrhosis decompensation (FIG. 3C) and this was most marked in a patient with ACLF who deteriorates on organ support (FIG. 3D).

### Example 2—HRV and Survival

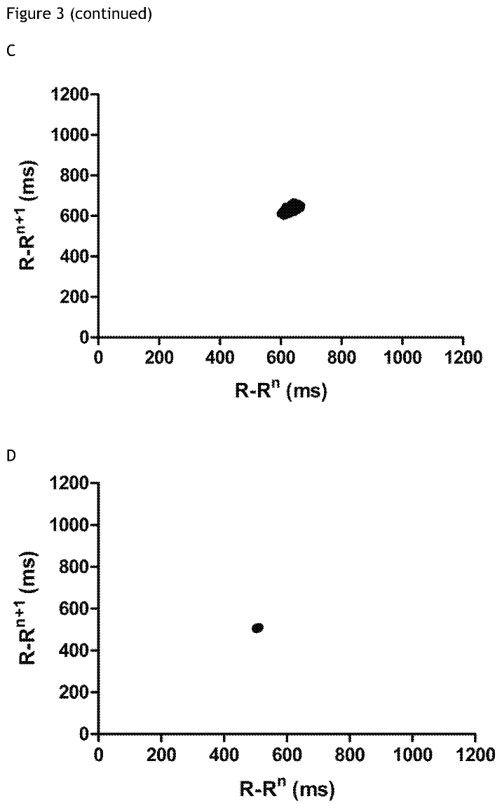

Baseline SDNN demonstrated good predictive utility for overall mortality within 3 months for pateints having or at risk of having AD: AUROC 0.75 (CI: 0.58-0.92), (FIG. 4). Using a SDNN cut-off of <12 ms derived from the ROC curve, established an optimal sensitivity of 88% and a negative predictive value for death of 93% for SDNN≥12 ms.

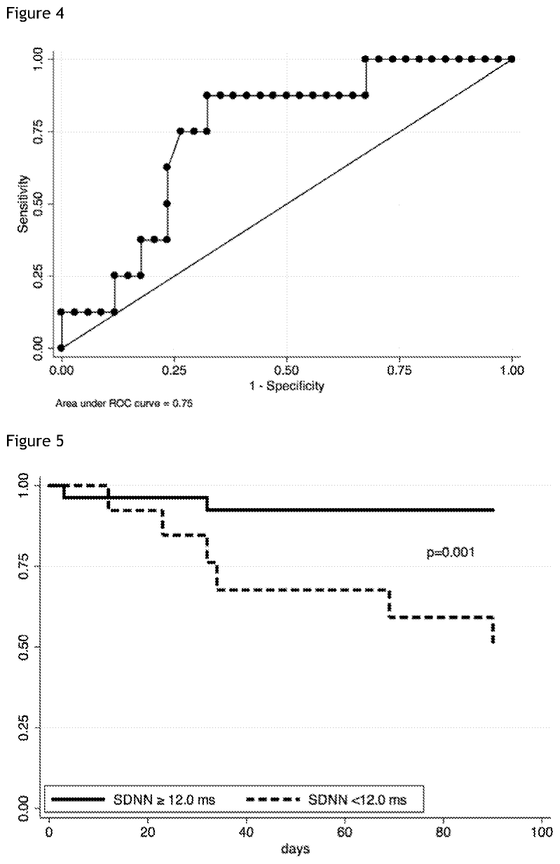

FIG. 5 shows a Kaplan-Meier analysis in which a cut-off for SDNN <12 ms highlights those patients who had markedly greater 3 month mortality of 40% vs. 8% in those with SDNN≥12 ms, P=0.001.

The association of SDNN with mortality does not appear confounded by other factors with no clear association between death and Child-Pugh score (p=0.12), MELD-Na score (p=0.56), CLIF-AD score (p=0.20).

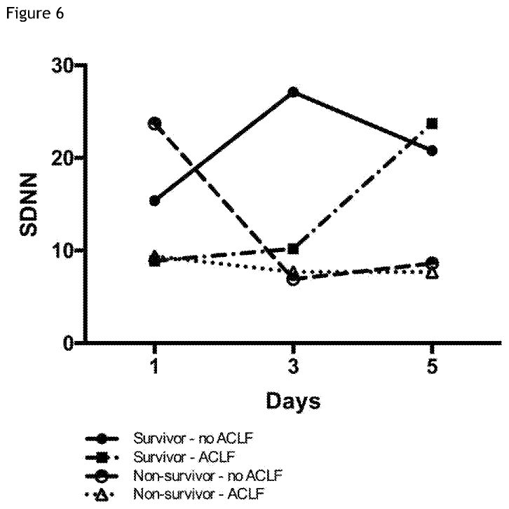

FIG. 6 shows representative plots of acute decompensated cirrhosis patients with repeated measures of SDNN over 5 days, during their episode of acute decompensation. Patients were separated by whether they develop organ failure and full fill criteria for ACLF vs. those that stay as AD without organ failure. Patients that develop ACLF have lower baseline SDNN than patients without ACLF. Moreover, SDNN deteriorates in patients that died and increases in survivors, suggesting a potential for dynamic testing of SDNN, to evaluate risk of poor outcome in a given patient.

Furthermore, for patients who recovered from their episode of acute decompensation and were discharged from the hospital, 67% of these patients incurred readmission (median 109 days) with new decompensation events (75% with multiple admissions), if they had SDNN <19.65 ms.

### Example 3—Correlation with Inflammation (CRP and WBC)

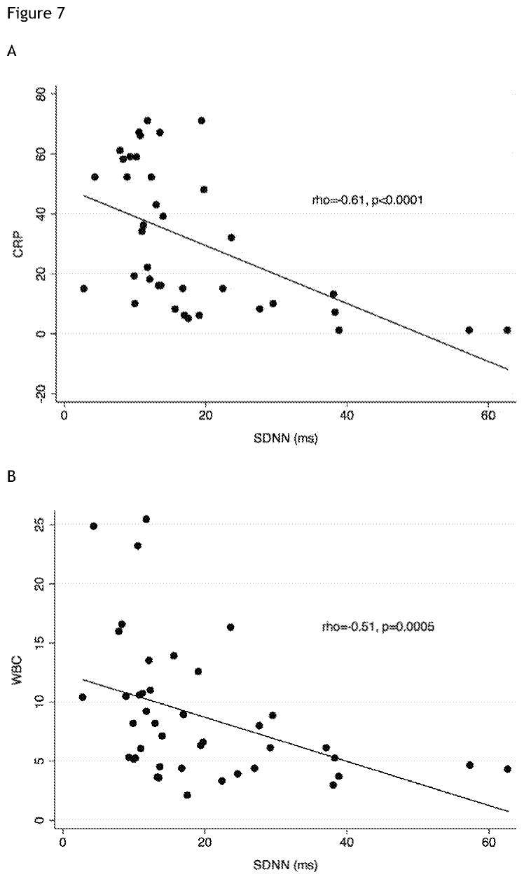

Baseline SDNN showed a strong inverse correlation with markers of inflammation including C-reactive protein (CRP) (rho −0.61, p<0.0001) and white cell count (WCC) (rho −0.51, p=0.0005) in patients having ACLF or at risk of developing ACLF (FIG. 7). There were similar trends in correlations with inflammatory cytokines (IL6: p=0.05, IL8: p=0.07).

### Discussion

A loss of HRV, as a manifestation of autonomic dysfunction, has been described in many conditions including cardiac ischaemia, diabetes mellitus, sepsis and chronic liver disease.

The key findings of the present invention are:

(1) Continuous, wireless remote monitoring of HRV is feasible in patients with AD or ACLF, or at risk of developing ACLF, and measuring SDNN reflects changes in HRV in this population.
(2) SDNN is reduced significantly with cirrhosis progression to acute decompensation and even further reduced in patients with ACLF.
(3) Reduction in SDNN, reflecting loss of HRV, negatively correlates with disease severity scores such as MELD and CP score and importantly, with systemic inflammation.
(4) Measurement of SDNN shows utility in predicting 90-day mortality after acute decompensation of cirrhosis and ACLF and an SDNN cut-off of ≥12 ms has a negative predictive value of 93% in determining outcome. Monitoring HRV, for example by measuring SDNN, is surprisingly an independent predictor of 90-day mortality in patients with, or at risk of having, AD or ACLF, whilst also highlighting patients likely to be readmitted with new cirrhosis decompensation.

1. A method of predicting acute decompensation (AD) and/or the development of acute-on-chronic liver failure (ACLF) in a patient having the condition, or at risk of having the condition, using heart-rate variability (HRV) as a marker.

2. The method of claim 1, wherein the method comprises predicting the severity of AD or ACLF in a patient having the condition, or at risk of having the condition, using HRV as a marker.

3. The method of claim 2, wherein the severity of AD or ACLF is defined as the likelihood of early mortality, wherein early mortality is a 90-day mortality or preferably a 28-day to a 90-day mortality.

4. A method of monitoring a patient with acute decompensation of cirrhosis or acute-on-chronic liver failure (ACLF) for prognosis of survival, which comprises:
(a) obtaining an initial measurement for heart rate variability (HRV);
(b) obtaining one or more subsequent measurements of HRV of the patient and determining whether said subsequent measurements fit any of predetermined criteria showing progression and/or severity of cirrhosis or acute-on-chronic liver failure (ACLF);
further optionally comprising a step (c) outputting an alarm if said measurements reaches a predetermined threshold for intervention.

5. The method of claim 1, comprising the use of a device capable of remotely monitoring HRV in the patient.

6. The method of claim 5, wherein the remote monitor is wireless.

7. The method of claim 1, wherein HRV is monitored by measuring the standard deviation of all normal beat-to-beat variation of R-R intervals (SDNN).

8. The method of claim 1, wherein a SSDN value of 12 ms or less is indicative of early mortality in patients having, or at risk of having, ACLF, wherein early mortality is a 90-day mortality or preferably a 28-day to a 90-day mortality.

9. The method of claim 1, further comprising a step of providing intervention in patients with, or at risk of having, AD or ACLF, based on the outcome of the method according to claim 1.

10. A database comprising information relating to HRV and its association with the likelihood of early mortality in patients having, or at risk of having, AD or ACLF, wherein early mortality is a 90-day mortality or preferably a 28-day to a 90-day mortality.

11. A method of determining the likelihood of 90-day mortality in a patient having, or at risk of having AD or ACLF comprising:
(a) inputting to a computer system data concerning the HRV of the patient;
(b) comparing the data to a computer database, which database comprises information relating to HRV and its association with the likelihood of early mortality in patients having, or at risk of having, AD or ACLF; and
(c) determining on the basis of the comparison the likelihood of early mortality in the patient;
wherein early mortality is a 90-day mortality or preferably a 28-day to a 90-day mortality.

12. A computer program comprising program code means that, when executed on a computer system, instruct the computer system to perform all the steps of claim 11.

13. A computer storage medium comprising the computer program according to claim 12 and a database comprising information relating to HRV and its association with the likelihood of early mortality in patients having, or at risk of having, AD or ACLF, wherein early mortality is a 90-day mortality or preferably a 28-day to a 90-day mortality.

14. A computer system arranged to perform the method according to claim 11, comprising:
(a) means for receiving data concerning the HRV of patients with, or at risk of having, AD or ACLF;
(b) a database comprising information relating to HRV and its association with the likelihood of early mortality in patients with, or at risk of having, AD or ACLF;
(c) a module for comparing the data with the database; and
(d) means for determining on the basis of said comparison the likelihood of early mortality in the patients;
wherein early mortality is a 90-day mortality or preferably a 28-day to a 90-day mortality.
